(19) United States
(12) Patent Application Publication
(10) Pub. No.: US 2025/0256013 A1
(43) Pub. Date: Aug. 14, 2025

(54) ANTIMICROBIAL COMPOSITIONS AND ARTICLES MADE THEREFROM

(71) Applicant: Solventum Intellectual Properties Company, Maplewood, MN (US)

(72) Inventor: Vinod P. Menon, Woodbury, MN (US)

(21) Appl. No.: 19/119,944

(22) PCT Filed: Oct. 9, 2023
(86) PCT No.: PCT/IB2023/060126

§ 371 Date: Apr. 10, 2025

Related U.S. Application Data
(60) Provisional application No. 63/379,348, filed on Oct. 13, 2022.

(51) Int. Cl. A61L 31/16; A61L 31/10

(52) U.S. Cl. CPC A61L 31/16; A61L 31/10; A61L 2300/104; A61L 2300/206; A61L 2300/208; A61L 2300/404

(57) ABSTRACT

An antimicrobial-impregnated adhesive sheet includes an antimicrobial and a pressure-sensitive adhesive. The sheet includes a first major surface and a second major surface. The sheet includes a plurality of surface depressions extending inwardly from the first major surface. An initial available fraction of antimicrobial at the first major surface is at least 30%, based on the total amount of antimicrobial in the antimicrobial-impregnated adhesive sheet. The sheet exhibits a retention of the initial available fraction of antimicrobial at the first major surface, after accelerated aging, of at least 50%.

### BACKGROUND

Antiseptic-impregnated adhesives are discussed in, for example, U.S. Pat. Nos. 4,323,557, 9,713,659, and 9,764,059.

### DETAILED DESCRIPTION

Despite advances made in infection-control practices, surgical-site infections (SSIs) remain a substantial cause of morbidity, prolonged hospitalization, and death. In fact, SSIs are associated with a mortality rate of 3% and 75% of SSI-related deaths are directly attributable to the SSI. Surgeons currently rely on surgical drapes having iodine-impregnated adhesives to mitigate contact with pathogenic microbes. While povidone-iodine is a widely effective antiseptic, there are drawbacks to its use. For example, povidone-iodine may cause skin irritations in some individuals, and use with large wounds may lead to kidney problems, high blood sodium, and metabolic acidosis. Furthermore, the use of povidone-iodine is not recommended for those that are less than 32 weeks pregnant, those that are prescribed lithium, or those with thyroid problems. Chlorhexidine gluconate and octenidine hydrochloride are viable alternatives that are not associated with the aforementioned fallbacks.

Developing antiseptic-impregnated adhesives (e.g., chlorhexidine gluconate- and octenidine hydrochloride-impregnated adhesives) would help reduce surgical-site infection rates, meanwhile potentially avoiding the side effects associated with povidone-iodine. Consequently pressure-sensitive adhesives capable of carrying and delivering chlorhexidine gluconate, octenidine hydrochloride, or the like are desirable.

Efforts to develop antiseptic-impregnated adhesives have been met with significant challenges. Chlorhexidine gluconate (“CHG”) and octenidine hydrochloride (“octenidine”), each being highly polar compounds, tend to precipitate from hydrophobic adhesive compositions. The lack of CHG solubility or octenidine solubility in the adhesive effectively immobilizes CHG or octenidine such that it is unavailable for adequate transfer to a surface. Moreover, blending additives like CHG and CHG-solubilizing vehicles often compromise the strength of the adhesive, which leads to premature adhesive failure. In terms of surgical drapes, this premature adhesive failure is called ‘drape drift.’ When surgical drapes move or ‘drift,’ the patient experiences a greater exposure to microbes and becomes more vulnerable to infection.

The present disclosure is directed toward water soluble antimicrobial-based (e.g., chlorhexidine gluconate-based or octenidine hydrochloride-based) compositions for inclusion within pressure-sensitive adhesive (PSA) formulations, and medical articles made therefrom. Initially, it was believed that hydrophilic (polar) vehicles were required to render chlorhexidine gluconate or octenidine hydrochloride compatible with adhesives. It was later found that hydrophobic (non-polar) vehicles having vicinal (i.e., separated by two atoms; adjacent), or otherwise proximate (i.e., separated by three atoms), hydrogen-bonding groups were effective at solubilizing chlorhexidine gluconate and octenidine hydrochloride, which in turn compatibilized the hydrophobic chlorhexidine gluconate solutions and hydrophobic octenidine solutions with hydrophobic pressure-sensitive adhesives (see WO 2014/035981). Still later it was found that chlorhexidine gluconate and octenidine, despite being polar compounds, are readily soluble in hydrophobic plasticizers that have hydrogen-bonding groups spaced more than three atoms apart, and that hydrophobic vehicles bearing vicinal hydrogen-bonding groups can be detrimental to adhesive integrity as compared to compositions that are void of said hydrophobic vehicles.

It was then discovered, however, that compositions having such hydrophobic plasticizers, while initially providing adequate availability of the chlorhexidine gluconate or octenidine at the working surface of the adhesive (that is, the surface of the adhesive that is intended to be nearest the skin of the patient) (the “available surface concentration”), dropped significantly over time in storage. To address this deficiency, compositions and methods that include PSAs having certain HLB values and aqueous-antimicrobial components having heightened water concentrations were found to produce antiseptic impregnated PSAs with very high initial available surface concentration of chlorhexidine gluconate or octenidine at the working surface of the adhesive. Yet still, while an improvement relative to prior compositions, over time in storage, in some instances, these initially very high antiseptic availability levels were found to decrease to undesirably low levels.

Consequently, compositions and methods for minimizing the reduction over time in available antiseptic surface concentration, remain desirable.

In this work, surprisingly, it was discovered that the combination of:
(i) a PSA having certain HLB values; and
(ii) an aqueous-antimicrobial component (e.g., aqueous chlorhexidine salt component) having (a) a certain level of water; and (b) a water soluble complexing agent;
in combination with a mixing technique whereby the aqueous-antimicrobial component is combined with a solvent adhesive solution a relatively short period before coating the mixture onto a substrate (e.g., adhesive backing or release liner) can produce antimicrobial-impregnated adhesives (i) having very high initial working surface concentrations of the antimicrobial component and (ii) having the ability to retain or substantially retain such high working surface concentrations over time in storage. It was further discovered that that presence of these very high working surface concentrations (and, as will be discussed in greater detail below, the associated surface features at or near the working surface) did not negatively impact the adhesive properties of the antimicrobial-impregnated adhesives even under fluid challenge.

As used herein, “acid” refers to a carboxylic acid group, i.e., —CO₂H.

As used herein, “complexing agent” refers to a compound having at least two hydrogen-bonding functionalities and is capable of cooperative intermolecular hydrogen bond formation. The cooperative effect implies an inherent ability to form multiple hydrogen bonds.

As used herein, “disinfecting” refers to a reduction in the number of active microorganisms present on a surface being disinfected. Disinfecting may kill or prevent microorganisms from growing or proliferating.

As used herein, “hydrophilic-lipophilic balance” or “HLB” values are calculated using the method of Griffin (Griffin WC; J. Soc. of Cosmetic Chemists 5, 259 (1954)). Thus, as used herein, the “HLB Method” involves a calculation based on the following:

HLB=20*Mₕ/M

where Mₕ is the molecular mass of the hydrophilic portion of the molecule, and M is the molecular mass of the whole molecule, giving a result on a scale of 0 to 20. An HLB value of 0 corresponds to a completely lipophilic/hydrophobic molecule, and a value of 20 corresponds to a completely hydrophilic/lipophobic molecule.

As used herein, “plasticizer” refers to a substance or combination of substances that lowers the glass transition temperature of another substance (e.g., a pressure-sensitive adhesive). Plasticizers effectively soften, increase flexibility, increase plasticity, decrease viscosity, and/or decrease friction of a substance to which it is added.

As used herein, “polymer” refers to a substance having one or more repeating monomer units. The chemical identities of the polymeric substances herein are at times described in terms of the monomers to which the polymer is derived. A skilled artisan would readily understand the reactivity profile of the recited monomers and how the monomers could synthetically be joined to form the polymer.

As used herein, “pressure-sensitive adhesive” refers to a non-reactive, self-stick adhesive that forms a bond when pressure is applied. No solvent, water, or heat is required to activate a pressure-sensitive adhesive.

When referring to “solubility,” or “to solubilize” it should be understood that the solubility of a component A in a component B refers to conditions in which only component A and component B are present, e.g., no added salts, compounds, or the like. Furthermore, any solubility values provided herein are with regard to a temperature range of about 20° C. to about 23° C. at atmospheric pressure (i.e., 760 mm/Hg).

In some embodiments, the present disclosure is directed to antimicrobial adhesives. The antimicrobial adhesives may be formed by the combination of a solvent-based pressure sensitive adhesive solution and an aqueous antimicrobial composition.

In some embodiments, the solvent-based pressure sensitive adhesive solution may include a solvent and a pressure sensitive adhesive. Suitable solvents may include any organic solvent that is miscible with the pressure-sensitive adhesive. For example, the solvent may include ethyl acetate, heptane, toluene, and methyl ethyl ketone, propyl acetate, butyl acetate, acetone, methyl propyl ketone, methyl isobutyl ketone, dimethylacetamide, dimethylformamide, dimethyl sulfoxide, N-methylpyrrolidone, hexanes, petroleum ether, tetrahydrofuran, lower alcohols, glycol ethers, xylenes, a combination thereof, or the like.

In some embodiments, the pressure-sensitive adhesive may be selected from an acrylic polymer or copolymer. In some embodiments, the acrylic polymer or copolymer may be the reaction product one of monomers selected from an alkyl (meth)acrylate, N-vinyl pyrrolidone, N-vinyl caprolactam, (alkyl-substituted)acrylamide, (alkyl-substituted)methacrylamide, 2-hydroxyethyl (meth)acrylate or and a combination thereof. In some embodiments, the pressure-sensitive adhesive may be selected from a rubber polymer or copolymer. Suitable rubber polymers include natural rubber, polybutadiene, and polyisobutylene. Suitable rubber copolymers include styrenic block copolymers such as styrene-butadiene-styrene, styrene-isoprene-styrene, and styrene-ethylene-butadiene-styrene. In some embodiments, the pressure-sensitive adhesive may be selected from a tackified silicone polymer.

In some embodiments, the pressure-sensitive adhesive may be characterized by a glass transition temperature (T_{g}) of about −70° C. to about 20° C. In some embodiments, the pressure-sensitive adhesive may be characterized by a T_{g} in ° C. of about −70, −60, −50, −40, −30, −20, −10, −5, 0, 5, 10, or 20, or a value within a range between any of the preceding values, for example, between about −20 and about 5, between about −50 and about −30, or the like.

As discussed above, it was discovered that pressure sensitive adhesives having HLB values within a certain range may, in part, contribute to the formation of antimicrobial adhesives having very high working surface antimicrobial concentrations. Generally, it is believed that such certain HLB values contribute to the generation of discrete secondary phase regions, discussed in greater detail below, that are relatively large. In this regard, in some embodiments, the pressure sensitive adhesives may have an HLB value that is less than 5, less than 4.6, or less than 4.5.

In some embodiments, pressure sensitive adhesive solution may include pressure sensitive adhesive in an amount of 10 wt.-%, 15 wt.-%, 20 wt.-%, 25 wt.-%, 30 wt.-%, 35 wt.-%, 40 wt.-%, 45 wt.-%, 50 wt.-%, or a value within a range between any of the preceding values, for example, between 15 and 50 wt.-% or 15 and 30-wt.-%, based on the total weight of the pressure sensitive adhesive solution. In some embodiments, pressure sensitive adhesive solution may include solvent in an amount of 30 wt.-%, 40 wt.-%, 50 wt.-%, 60 wt.-%, 70 wt.-%, 80 wt.-%, 90 wt.-%, 95 wt.-%, or a value within a range between any of the preceding values, for example, between 50 and 90 wt.-% or 50 and 80-wt.-%, based on the total weight of the pressure sensitive adhesive solution.

In some embodiments, in addition to pressure sensitive adhesive and solvent, the pressure sensitive adhesive solution may include a plasticizer. Any suitable plasticizer may be used as long as it does not unacceptably affect the properties of the pressure sensitive adhesive solution or the PSA made therefrom. Such a plasticizer may be optimally selected to be compatible with (i.e., miscible with) the other components in the adhesive composition. Potentially suitable plasticizers include various esters, e.g. adipic acid esters, formic acid esters, phosphoric acid esters, benzoic acid esters, phthalic acid esters, esters of dimer diacids with dimer diols; sulfonamides, and naphthenic oils. Other potentially suitable plasticizers include e.g. hydrocarbon oils (e.g., those that are aromatic, paraffinic, or naphthenic), vegetable oils, hydrocarbon resins, polyterpenes, rosin esters, phthalates, phosphate esters, dibasic acid esters, fatty acid esters, polyethers, and combinations thereof; plant fats and oils such as olive oil, castor oil, and palm oil; animal fats and oils such as lanolin; fatty acid esters of polyhydric alcohols such as a glycerin fatty acid ester and a propylene glycol fatty acid ester; and, fatty acid alkyl esters such as ethyl oleate, isopropyl palmitate, octyl palmitate, isopropyl myristate, isotridecyl myristate, and ethyl laurate, esters of a fatty acid. Any of the above plasticizers may be used alone or in combination (and/or in combination with any other additive mentioned herein). Plasticizer, if present, may be present in an amount of 10 wt.-%, 15 wt.-%, 20 wt.-%, 25 wt.-%, 30 wt.-%, 35 wt.-%, 40 wt.-%, 45 wt.-%, 50 wt.-%, or a value within a range between any of the preceding values, for example, between 10 and 50 wt.-% or 15 and 30-wt.-%, based on the total weight of the pressure sensitive adhesive solution.

In various embodiments, the aqueous antimicrobial composition may include one or more antimicrobials, water, and one or more water soluble complexing agents.

In some embodiments, suitable antimicrobials may include any antimicrobials that are selectively soluble in water (i.e., that are soluble in a minor/water phase but not in a major/organic phase). It is noted that the flexibility in suitable antimicrobials represents an advantage of the antimicrobial adhesives of the present disclosure. That is, while prior antimicrobial adhesives were formulated to specifically exclude water by drying water from a commercial aqueous antimicrobial drug prior to incorporation, predissolving the drug in a hydrophobic plasticizer, or utilizing organic soluble antimicrobials, the concepts and advantages of the present disclosure may be accomplished with any selectively water-soluble antimicrobial. In some embodiments, suitable water-soluble antimicrobials (and that are selectively soluble in water) may include chlorhexidine gluconate (CHG), chlorhexidine acetate, octenidine hydrochloride, polyhexamethylene biguanide salts (PHMB), quat ammonium salts, chlorhexidine salts, silver salts, water-soluble iodophors, triclosan, or combinations thereof.

In some embodiments, water-soluble antimicrobials may be present in the aqueous antimicrobial composition in an amount of at least about 0.05 wt-%, based on the total weight of the aqueous antimicrobial composition. In some embodiments, water-soluble antimicrobials may be present in the aqueous antimicrobial composition in an amount of no more than about 5 wt-%, based on the total weight of the aqueous antimicrobial composition. In some embodiments, water-soluble antimicrobials may be present in the aqueous antimicrobial composition in an amount (wt-% with respect to the total weight of the aqueous antimicrobial composition.) of about 0.05, 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, or a value within a range of any of the preceding values, for example, between about 0.2 and about 4.0 or between about 2.0 and about 3.0.

In some embodiments, the aqueous antimicrobial composition may include water. As discussed above, it was discovered that the presence of water within a certain range that is higher than that which has been conventionally employed with respect to antimicrobial adhesives may, in part, contribute to the formation of antimicrobial adhesives having very high working surface antimicrobial concentrations. More specifically, and as will be discussed further below, it was discovered that the presence of water at higher concentrations than conventionally employed, at least in part, contributes to the formation of larger discrete aqueous phase regions (e.g., droplets) upon combination of the solvent-based pressure sensitive adhesive solution and the aqueous antimicrobial composition. In this regard, in some embodiments, water may be present in the aqueous antimicrobial composition in an amount of between 20 and 80 wt-%, between 30 and 70 wt-%, or between 40 and 60 wt-%, based on the total weight of the aqueous antimicrobial composition.

In some embodiments, the aqueous antimicrobial composition may include one or more water-soluble complexing agents. Generally, it was discovered that inclusion of such complexing agents contributed to a marked decrease in the reduction (or increase in retention) over time of available surface concentration. It is believed that such reduction may be attributable to the complexing agent's ability to, after dry down of the composition that results from combining the above-described solvent-based pressure sensitive adhesive solution and the aqueous antimicrobial composition, bind to the antiseptic and inhibit its movement into the bulk of the adhesive. Suitable water-soluble complexing agents may include any water-soluble compound falling within the above-provided definition of complexing agent. In some embodiments, suitable water-soluble complexing agents may include monomeric polyhydroxylated compounds (charged or uncharged) (e.g., glycerol, erythritol, sorbitol, xylitol, sodium gluconate, maltitol, trehalose), polymeric polyhydroxylated compounds (e.g., polyglycerol, poly(vinyl alcohol), dextrins, cyclodextrins, partially hydrolyzed poly(vinyl acetate), carrageenans, polyethylene glycol, starch, hyaluronic acid, xanthans, polyglycitol), carbamides (e.g., urea, hydroxyethyl urea, hydroxypropyl urea), lactams (e.g., poly(vinyl pyrrolidone), poly(N-vinyl caprolactam)), amides (e.g., acetamide, propionamide, butyramide), or combinations thereof.

In some embodiments, water soluble complexing agents may be present in the aqueous antimicrobial composition in an amount of between 0.1 and 10 wt-%, between 0.2 and 5 wt-%, or between 0.5 and 2 wt-%, based on the total weight of the aqueous antimicrobial composition.

In some embodiments, in addition to water and antimicrobials, the aqueous antimicrobial composition may include one or more co-solvents. Generally, any water miscible solvent may be employed as a cosolvent. Suitable cosolvents may include methanol, ethanol, isopropanol, butanol, acetone, tetrahydrofuran, dimethylformamide, dimethyl sulfoxide, glycol ethers, or the like. Co-solvents, if present, may be present in an amount of 20 wt.-%, 30 wt.-%, 40 wt.-%, 50 wt.-%, 60 wt.-%, 70 wt.-%, 80 wt.-%, or a value within a range between any of the preceding values, for example, between 20 and 80 wt.-% or 30 and 50-wt.-%, based on the total weight of the aqueous antimicrobial composition.

In some embodiments, the antimicrobial adhesives of the present disclosure may further include one or more of a tackifier, antioxidant, pigment, reinforcing filler, cross-linker, or electrolyte.

In some embodiments, the antimicrobial adhesive may be characterized by a glass transition temperature (T_{g}) of about −90° C. to about 10° C. In some embodiments, the antimicrobial adhesive may be characterized by a glass transition temperature (° C.) of about −90, −80, −70, −60, −50, −40, −30, −20, −10, −5, 0, 5 or 10, or a value within a range between any of the preceding values, for example, between about −30 and about −5, between about −70 and about 0, or the like.

As discussed above, it was discovered the above-described solvent-based pressure sensitive adhesive solutions and aqueous antimicrobial compositions could be combined to produce antimicrobial-impregnated adhesives having very high working surface concentrations of the antimicrobial component. Further regarding this discovery, it was observed that when the above-described solvent-based pressure sensitive adhesive and aqueous antimicrobial compositions are combined, a two-phase composition is formed. More specifically, it was observed that a primary or major organic phase that includes at least the pressure sensitive adhesive and solvent, and a secondary or minor aqueous phase that includes at least water and the water-soluble antimicrobial, are formed. It was further observed that, in some embodiments, at least initially, the discrete aqueous phase regions are relatively large and tend to descend (in the direction of gravitational force) into the organic phase such that the discrete aqueous phase regions collect at or near a bottom surface of the organic phase (which, as discussed below, may correspond to the working surface of the adhesive).

In some embodiments, the discrete aqueous phase regions may have a shape that is spherical. In relation to the discrete aqueous phase regions (or discrete surface features as described below), “spherical” refers to a geometric shape that is perfectly spherical, or a spherical shape within ±10% or ±5% (that is, the eccentricity in either direction does not exceed 5% or 10%), or any portion of such geometric shape (e.g., hemispherical). In some embodiments, the discrete aqueous phase regions may have shapes that are spherical or elliptical.

In some embodiments, the discrete aqueous phase regions may have an average longest dimension (e.g., diameter) of between 4 and 100 micrometers, between 10 and 60 micrometers, or between 15 and 50 micrometers. Further regarding the size of the discrete aqueous phase regions, it was discovered that the higher the size of the discrete aqueous phase regions (e.g., droplets), the higher the probability that such discrete phase migrates through the organic phase and to a bottom surface thereof. Still further, it was discovered that the size of the discrete aqueous phase regions is a function of, at least in part, the amount of water that is present in the aqueous antimicrobial compositions (although it was also observed that increasing the amount of water beyond a certain point can cause coagulation of the pressure sensitive adhesive in the continuous organic phase).

It was further observed that after settling of the discrete aqueous phase regions into the organic phase and subsequent drying (i.e., removal or partial removal of water and solvent), the dried down equivalent of the discrete aqueous phase regions remains on the bottom surface of the pressure sensitive adhesive as depressions or dimples (having the same or substantially the same shape, size, and distribution of the discrete aqueous phase regions prior to drying) having the antimicrobial contained therein (likely in the form of a coating that coats the surface of the depressions).

In this regard, in some embodiments, the present disclosure is directed to antimicrobial-impregnated adhesives that are formed from drying down the composition that results from combining the above-described solvent-based pressure sensitive adhesive solution and the aqueous antimicrobial composition, and which have been combined utilizing the techniques of the present disclosure (e.g., a technique whereby the aqueous-antimicrobial component is combined with the solvent adhesive a relatively short period before the combined composition is coated onto a substrate). Such antimicrobial-impregnated adhesives may have any one, any combination, or all of the following properties:
Discrete surface depressions (e.g., dimples)
Uniform or substantially uniform distribution of surface depressions
Antimicrobial material in and/or around the depressions
An initial available fraction of antimicrobial at the working surface (which can be considered as, within a relatively short period after formation of the adhesive sheet (e.g., within 24 hours), the amount of antimicrobial that is available for antimicrobial action at the adhesive working surface-contact surface of the patient (e.g., skin) interface), of at least 30%, at least 40%, at least 50%, at least 60%, at least 70%, or at least 80%, based on the total amount of antimicrobial in the adhesive. For purposes of the present disclosure, the initial available fraction of antimicrobial at the working surface is as determined in accordance the Available Fraction Test of the Examples.
A rate of antimicrobial release (which can be considered as the time required for release of the available antimicrobial microbial upon exposure to an aqueous solution/the contact surface of a patient), of no more than 10 minutes, no more than 7 minutes, or no more than 5 minutes. For purposes of the present disclosure, the rate of antimicrobial release is as determined in accordance the Rate of Release Test of the Examples.
A retention of at least 50%, at least 60%, at least 70%, or at least 80%, at least 90%, or at least 100% of the initial available fraction of antimicrobial at the working surface after accelerated aging. For purposes of the present disclosure, accelerated aging refers to the accelerated aging conditions of the Examples—elevated humidity and elevated temperature for at least 3 months).

For purposes of the present disclosure, the percent retention of the initial available fraction of antimicrobial at the working surface is determined using the Available Fraction Test of the Examples (and comparing the initial available fraction to the available fraction after accelerated aging).

Further regarding the discrete surface depressions, in some embodiments, the antimicrobial-impregnated adhesives of the present disclosure may be characterized as adhesive sheets having at least one topologically microstructured major surface that includes a plurality of micro-dimples or micro-craters protruding inwardly from the major surface, and which are distributed uniformly (or substantially uniformly) about such major surface. As discussed above, the surface depressions may generally conform to the discrete aqueous phase regions prior to drying of the antimicrobial-impregnated adhesive.

In some embodiments, the discrete surface depressions (or micro-dimples or micro-craters) may have an average size (in terms of average longest dimension) of between 4 and 100 micrometers, between 10 and 60 micrometers, or between 15 and 50 micrometers. In some embodiments, the discrete surface depressions may be uniformly distributed such that the number of discrete surface depressions about the major surface, per square millimeter, differs by no more than 10% or no more than 5%. As described above, the discrete surface depressions may have a shape that is spherical. In some embodiment, the regions of the working surface of the adhesive having discrete surface depressions may account for between 3 and 40%, between 5 and 20%, between 6 and 18%, between 7 and 15%, or between 8 and 12%, of the total surface area of the major surface of the adhesive sheet (the total surface area being made up of the discrete surface depressions and the planar (or nearly planar) regions that extend between discrete surface depressions. It is noted that in the present work, it was confirmed that presence of discrete surface depressions at the adhesive working surface-contact surface of the patient (e.g., skin) interface did not negatively impact adhesive properties even under fluid challenge.

In various embodiments, a medical article is described. The medical article may include a substrate (or adhesive carrier) having a first surface and second surface opposite the first surface, and any antimicrobial adhesive described herein disposed on the first surface.

In some embodiments, the substrate (or adhesive carrier) may be a polymeric film. The polymeric film may be woven or nonwoven.

In some embodiments, the medical article may further include a release liner in contact with the antimicrobial adhesive. The release liner may protect the antimicrobial adhesive from coming into contact with foreign matter prior to use. The release liner may further facilitate application of the medical article to a surface. In some embodiments, the major surface of the antimicrobial adhesive having a high concentration of antimicrobial may be nearest the release liner (as opposed to the substrate). In this regard, once the release liner is removed and the antimicrobial adhesive applied to the skin of the patient, the major surface of the antimicrobial adhesive having a high concentration of antimicrobial may be in contact with the skin of the patient.

In some embodiments, the medical article may further include a delivery system disposed on the second surface. The delivery system may be, for example, a paper that is releasably secured to the second surface with an adhesive. The delivery system may provide structural integrity to the medical article in order to facilitate application of the medical article to a surface.

In some embodiments, the medical article may be configured in various shapes, including custom shapes for fitting over contoured surfaces.

In some embodiments, the medical article may be in the form of a sheet or a roll.

In some embodiments, the antimicrobial article is a tape or wrap.

In some embodiments, the medical article is a wound dressing. The medical article may be in the shape of any wound dressing known in the art.

In some embodiments, the medical article is an intravenous dressing.

In some embodiments, the medical article is a surgical drape.

In various embodiments, a method for preparing the antimicrobial adhesives described herein is provided. The method may include contacting an aqueous antimicrobial composition described herein and a solvent-based pressure sensitive adhesive solution described herein to form an antimicrobial adhesive precursor. In some embodiments, the step of contacting aqueous antimicrobial composition and a solvent-based pressure sensitive adhesive solution may include, for example, combining the two parts via a mixing tube as described in U.S. Pat. No. 3,865,352, which is herein incorporated by reference in its entirety, or by combining the two parts in a suitable vessel and then maintaining the suspension through constant agitation. Of course, any other conventional methods of combining components of a mixture may be employed without deviating from the scope of the present disclosure.

In some embodiments, the methods of the present disclosure may then include, shortly after the step of contacting the aqueous antimicrobial composition and the solvent-based pressure sensitive adhesive solution, depositing (e.g., coating) the resulting composition (or antimicrobial adhesive precursor or wet pressure sensitive adhesive) onto a substrate (e.g., a release liner or carrier). Generally, it was observed that if too much time elapses between the step of contacting and the step of depositing the antimicrobial adhesive precursor (may alternatively be referred to as a wet pressure sensitive adhesive), the discrete aqueous phase regions can collapse into the bulk, or major organic phase (as opposed to collecting near a bottom surface of the major organic surface) (the useful “pot-life” of the suspension is also dependent on the viscosity of the solvent-based pressure sensitive adhesive with higher viscosity solutions generally affording higher suspension stability). In this regard, in some embodiments, the methods of the present disclosure may include depositing the antimicrobial adhesive precursor within 30 minutes, within 10 minutes, or within 2 minutes of contacting the aqueous antimicrobial composition and the solvent-based pressure sensitive adhesive solution. In some embodiments, depositing the antimicrobial adhesive onto the substrate may include any conventional deposition technique such as such as dip coating, knife coating, extrusion coating, spin coating, slide hopper coating, curtain coating, or the like.

In some embodiments, the antimicrobial adhesive precursor may be deposited onto the substrate at a generally uniform thickness. In some embodiments, the antimicrobial adhesive precursor may be deposited onto the substrate at a thickness of between 5 micrometers and 100 micrometers, between 10 micrometers and 50 micrometers, or between 15 micrometers and 40 micrometers. The antimicrobial adhesive precursor may be deposited onto the substrate as a continuous coating or a series of discrete or patterned coatings.

In some embodiments, the method may further include drying (to reduce or remove solvent and water) the antimicrobial adhesive precursor to form the antimicrobial adhesive. In some embodiments, the step of drying may include one or more of heating; vacuum, including roto-evaporating or freeze-pump-thaw techniques, distillation or azeotropic distillation, molecular sieves, or the like. In some embodiments, drying may include heating the precursor to a temperature (° C.) of 30, 40, 50, 60, 70, 80, 90, 100, or a value within a range of any of the preceding values, for example, between 40 and 60, between 50 and 70, or the like. In some embodiments, the heating may be conducted under vacuum. In some embodiments, heating may be carried out for a period of about 1 min to about 10 min.

In some embodiments, after drying, the antimicrobial adhesive may be borne on a surface of the substrate at a thickness of (in microns) 50, 75, 100, 125, 150, 175, 200, 225, 250, 275, 300, 325, 350, 375, 400, 425, 450, 475, 500, or 525, or a value between any of the preceding values, for example, between 100 and 200, between 75 and 350, or the like.

In various embodiments, a method for preparing a medical article is provided. The method may include preparing an antimicrobial adhesive as described above, where the substrate is backing material or release liner for a medical article. In some embodiments, the substrate may be a polymeric backing material, such as thermoplastic polyurethanes (e.g., as sold by Lubrizol Inc. under the tradename ESTANE). In other embodiments, the substrate may be a release liner, which can be made from a variety of materials such as paper, poly-coated paper, polyester film, high-density polyethylene film, silicone, or the like. In embodiments wherein the substrate may be a polymeric backing material, the method may further include contacting the dried antimicrobial adhesive to a release liner. In embodiments where the substrate is a release liner, the method may further include laminating the dried antimicrobial adhesive to a polymeric backing material. The laminating may be performed using nip rollers at room temperature.

In various embodiments, a method for disinfecting a surface is described. The method may include providing a medical article described herein, and contacting the medical article to the surface for a period. In some embodiments, the surface may be skin or tissue. In some embodiments, the skin or tissue is mammalian skin or tissue. In some embodiments, the tissues may be selected from mucosal tissues, chronic wounds, acute wounds, burns, or the like. In some embodiments, the skin or tissue may be intact, i.e., undamaged. In some embodiments, the skin or tissue may be wounded or otherwise damaged. In some embodiments, the skin or tissue may be intact upon contacting and may remain in contact upon subjecting the skin to damage, e.g., cutting, piercing, or the like.

In other embodiments, the surface may be a medical surface, for example, surgical devices (e.g., scalpel, scissors, blades, forceps, drapes, or the like), medical devices (e.g., catheters, stents, artificial joints, dental implants, or the like), floor tiles, countertops, tubs, dishes, gloves, swabs, cloth, sponges, foams, nonwovens, and paper products.

In some embodiments, the medical articles may be effective against various microorganisms types, e.g., Gram positive bacteria, Gram negative bacteria, fungi, protozoa, mycoplasma, yeast viruses, lipid-enveloped viruses, or the like. For example, the antimicrobial adhesives and medical articles made therefrom may be effective at reducing the number of microorganisms present on the surface and/or preventing the growth of such microorganisms, e.g., Staphylococcus spp., Streptococcus spp., Pseudomonas spp., Enterococcus spp., Escherichia spp., Aspergillus spp., Fusarium spp., Candida spp., Staphylococcus aureus, methicillin-resistant Staphylococcus aureus (MRSA), Staphylococcus epidermidis, Streptococcus pneumoniae, Enterococcus faecalis, vancomycin-resistant Enterococcus (VRE), Pseudomonas aeruginosa, Esherichia coli, Aspergillus niger, Aspergillus fumigatus, Aspergillus clavatus, Fusarium solani, Fusarium oxysporum, Fusarium chlamydosporum, Candida albicans, Candid glabrata, Candida krusei, or the like.

In some embodiments, the medical article may contact the surface for a period in minutes of about 30, 60, 90, 120, 150, 180, or 210 or a value between any of the preceding values, for example, between about 30 and about 120, between about 90 and about 180, or the like. In other embodiments, the antimicrobial article may contact the surface for a period in hours of greater than about 1, 2, 3, 4, 5, 12, or 24, or a value between any of the preceding values, for example between about 2 and about 5, between about 12 and about 24, or the like. In some embodiments, the antimicrobial article may contact the surface for a period in days of about 1, 2, 3, 4, 5, 6, or 7, or a value between any of the preceding values, for example, between about 1 and about 2, between about 2 and about 5, or the like.

In some embodiments, the method may be effective to deliver any of the above-described antimicrobials or a combination thereof to the surface at an average rate of greater than 15 mcg/sq in per hour. For example, the method may be effective to deliver any of the above-described antimicrobials or a combination thereof to the surface at an average rate (mcg/sq. in per hour) of about 15, 20, 25, 30, 35, 40, 45, 50, 60, 70, 80, 90, or 100, or a value between any of the preceding values, for example, between about 30 and about 50, between about 20 and about 60, or the like.

In some embodiments, the method may be a method for preparing a surface for incision, e.g., surgery. In some embodiments, the method may be a method for preparing a surface for needle penetration, e.g., to administer intravenous pharmaceuticals or fluids, withdraw fluids, or the like.

In various embodiments, a kit is described. The kit may include a medical article described herein and a set of instructions directing a user to disinfect a surface according to the methods described herein.

In various embodiments, a kit is described. The kit may include a medical article described herein and a set of instruction directed a user to prepare a surface for surgery according to the methods described herein.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

These examples are merely for illustrative purposes only and are not meant to be limiting on the scope of the appended claims. All parts, percentages, ratios, etc. in the examples and the rest of the specification are by weight, unless noted otherwise. Solvents and other reagents used were obtained from MilliporeSigma, St. Louis, MO, unless otherwise noted. The following abbreviations are used: cm=centimeter; g=gram; nm=nanometer; ppm=parts per million. PSA refers to pressure sensitive adhesive. A mil is one thousandth of an inch. R.H. is used for relative humidity. The terms “weight %”, “% by weight”, and “wt %” are used interchangeably.

### Elution Test:

Antimicrobial elution from adhesive samples is measured by exposing the adhesive portion of each sample having 3.14 cm² area uniformly to 750 microliters of water for 30 minutes, diluting 3-fold in water, measuring the UV absorbance in a microcuvette at 254 nm of this dilute extract, and determining concentration from a calibration curve. The extracted concentration is re-expressed in units of 3.14 cm² extracted in 50 microliters.

### Available Fraction Test:

The available fraction is calculated by ratioing the weight of extracted antimicrobial in the Elution Test to the theoretical weight of antimicrobial present in 3.14 cm² area of coated adhesive. The coated adhesive is incubated for less than 24 hours at room temperature prior to the Elution Test. The weight of extracted antimicrobial is the initial available amount, and may be expressed as a fraction or weight percent of the theoretical weight of antimicrobial present. If the theoretical weight of antimicrobial present is 1000 mg and 500 mg is extracted, this provides an initial available fraction of 50%.

### Accelerated Aging Conditions:

Accelerating aging refers to samples exposed to elevated temperature and humidity for a desired period of time prior to testing. To simulate one year of shelf life, standard accelerating aging conditions for drug products are 40° C. and 75% relative humidity (40° C./75% R.H.) for three months. Acceptable product performance after exposure to these accelerated aging conditions predicts a shelf-life of one year at room temperature. Testing after exposure to the standard conditions of 40° C. and 75% relative humidity (40° C./75% R.H.) for three months may be referred to as accelerated aging testing.

### Incubation:

Incubation refers to holding samples at specified temperature and humidity for a specified time. The combination of temperature, humidity, and time used for incubations are not the conditions used for accelerated aging conditions.

### Retention:

Retention refers to the comparison of the elution of antimicrobial agent from samples that have undergone accelerated aging or incubation to samples that have not been subjected to accelerated aging or incubation. Retention may be calculated by dividing the initial available fraction of a sample exposed to accelerated aging or incubation to the initial available fraction of a sample that has not been subjected to accelerated aging or incubation. Retention may be expressed as a percentage.

TABLE 1
Materials.

| Material | Abbreviation | Source (Location) |
|---|---|---|
| Heptane | | MilliporeSigma |
| | | (St. Louis, MO) |
| Propyl acetate | | MilliporeSigma |
| | | (St. Louis, MO) |
| Poly(IOA/NVP) solution, | | 3M Company |
| 85/15 weight ratio, | | (St. Paul, MN) |
| 26.5% w/w in propyl acetate | | |
| Priplast 3197 | | Croda Inc. |
| | | (Edison, NJ) |
| Isopropanol | | MilliporeSigma |
| | | (St. Louis, MO) |
| Sorbitol | | MilliporeSigma |
| | | (St. Louis, MO) |
| Polyglycerol-3 | PG-3 | Solvay Interox |
| | | (Houston, TX) |
| Chlorhexidine gluconate | CHG | Xttrium Laboratories, |
| | | Inc. (Mount Prospect, IL) |
| Sodium gluconate | | MilliporeSigma |
| | | (St. Louis, MO) |
| Polyvinylpyrrolidone, | PVP-10k | MilliporeSigma |
| 10,000 g/mol | | (St. Louis, MO) |
| Urea | | MilliporeSigma |
| | | (St. Louis, MO) |
| Xylitol | | MilliporeSigma |
| | | (St. Louis, MO) |
| Polyvinylpyrrolidone, | PVP-K17 | BASF Corp. |
| 17,000 g/mol | | (Mt. Olive, NJ) |
| Polyvinylpyrrolidone, | PVP-K90 | BASF Corp. |
| 90,000 g/mol | | (Mt. Olive, NJ) |
| Ioban EZ polyethylene | | 3M Company |
| release liner | | (St. Paul, MN) |

### Example 1

This example demonstrates the utility of two different water-soluble complexing agents on the retention of CHG at the adhesive surface. For each sample type, the following parts A and B were made separately as provided in Table 2 and mixed immediately prior to coating at 9 mils wet thickness on an Toban EZ liner. The wet adhesive was dried at 170° F. and laminated to a 0.8 mil thick polyurethane film. In addition to a control adhesive, adhesive samples were made containing 1000 sorbitol and 20% polyglycerol-3 on a weight percent of solids basis.

TABLE 2
Adhesive formulations.

| | | % w/w | | |
|---|---|---|---|---|
| | Component | Control | 10% sorbitol | 2% PG-3 |
| Part A | Heptane | 25 | 25 | 25 |
| | Propyl acetate | 19.4 | 19.16 | 19.4 |
| | Poly(IOA/NVP) | 45.4 | 45.4 | 45.4 |
| | (85/15) solution | | | |
| | Priplast 3197 | 5.1 | 5.1 | 5.1 |
| | Total | 94.9 | 94.66 | 94.9 |

| | | % w/w | | |
|---|---|---|---|---|
| | Component | Control | 10% sorbitol | 2% PG-3 |
| Part B | Isopropanol | 2 | | 2 |
| | Sorbitol | | 3.5 | |
| | PG-3 | | | 0.7 |
| | Water | 2.75 | 1.49 | 2.05 |
| | Chlorhexidine gluconate | 0.36 | 0.36 | 0.36 |
| | Total | 5.1 | 5.35 | 5.1 |

A portion of the three coated samples was incubated for 3 days at 80° C./75% R.H. Specimens from each of the three sample groups were measured for CHG elution both freshly fabricated and after incubation. The freshly fabricated specimens served as controls. Antimicrobial elution from adhesive samples was measured by exposing the adhesive portion of each sample having 3.14 cm² area uniformly to 750 microliters of water for 30 minutes, diluting 3-fold in water, measuring the UV absorbance in a microcuvette at 254 nm of this dilute extract, and determining concentration from a calibration curve. The extracted concentration was re-expressed in units of 3.14 cm² extracted in 50 microliters. The results are provided in Table 3.

TABLE 3
CHG elution from freshly fabricated and incubated (3 d) adhesive samples.

| | Eluted CHG (ppm) | | | | | |
|---|---|---|---|---|---|---|
| Time | | Control | | 10% sorbitol | | 2% PG-3 |
| (minutes) | Control | (incubated) | 10% sorbitol | (incubated) | 2% PG-3 | (incubated) |
| 5 | 2671 | 1253 | 2731 | 2036 | 1413 | 1126 |
| 15 | 2802 | 1316 | 2825 | 2197 | 2052 | 2126 |
| 30 | 3038 | 1736 | 3038 | 2489 | | |

Both complexing agents were found to exert a protective effect on the CHG release from the surface of the adhesive. The results indicate that lower levels of complexing agent approaching the concentration of CHG are sufficient for protection.

### Example 2

This example expands the selection of complexing agents to demonstrate the generality of the effect. Ionic (sodium gluconate) and polymeric (Polyvinylpyrrolidone or PVP) complexing agents are included.

Control adhesive was made, coated and laminated as in Example 1. The complexed samples were made by adding the complexing agent to part B, replacing all of the water and part of the isopropanol for the 4% complexing agent samples. A part of the isopropanol was replaced as well for the 8% complexing agent sample. A portion of the laminated samples was incubated for 3 days at 80° C./75% R.H. and all the samples were tested for CHG elution as in Example 1 using an elution time of 30 minutes. The results are provided in Table 4.

Note that there is a steep drop in the control and 8% PVP sample. All the remaining complexing agents including PVP at the lower 4% exhibited a remarkable protective effect on stabilizing CHG at the adhesive surface.

TABLE 4
CHG elution from the samples of Example 2.

| | Eluted | S.D. |
|---|---|---|
| Sample | CHG (ppm) | (ppm) |
| Control, Initial | 3038 | |
| Control, 3 d, 80° C./75% R.H. | 1736 | |
| 4% sodium gluconate, Initial | 1848 | 154 |
| 4% sodium gluconate, 3 d, 80° C./75% R.H. | 2560 | 79 |
| 8% PVP-10k, Initial | 2784 | 53 |
| 8% PVP-10k, 3 d, 80° C./75% R.H. | 1522 | 291 |
| 4% Sorbitol, Initial | 2265 | 75 |
| 4% Sorbitol, 3 d, 80° C./75% R.H. | 2306 | 3 |
| 4% Urea, Initial | 2166 | 206 |
| 4% Urea, 3 d, 80° C./75% R.H. | 1932 | 98 |
| 4% PG-3, Initial | 2249 | 24 |
| 4% PG-3, 3 d, 80° C./75% R.H. | 2463 | 124 |
| 4% PVP-10k, Initial | 2488 | 31 |
| 4% PVP-10k, 3 d, 80° C./75% R.H. | 2722 | 193 |

### Example 3

This example demonstrates the protective effect of complexing agents when the experiment is conducted at lower incubation temperatures at the same elevated humidity as in the previous examples. The incubation was conducted for longer periods and additional concentrations of caging agent were explored.

Control adhesive was made, coated and laminated as in Example 1 The complexed samples were made by adding the complexing agent to part B, replacing all of the water and part of the isopropanol for the 4% complexing agent samples. Samples with the lower levels of complexing agents had only a portion of the water replaced. A portion of the laminated samples was incubated for 1 and 3 weeks at 40° C./75% R.H. and all the samples were tested for CHG elution as in Example 1 with an elution time of 30 minutes. The results are provided in Table 5.

At the lower temperatures and extended times, the reduced rate of CHG release continues unabated in the control. All of the complexing agents showed improved CHG release from incubated adhesive versus control. The PVP shows some drop-off at very low usage of 0.25%.

TABLE 5
CHG elution from the samples of Example 3.

| | Eluted | S.D. |
|---|---|---|
| Sample | CHG (ppm) | (ppm) |
| Control, Initial | 2590 | 52 |
| Control, 1 week, 40/75 | 1486 | 622 |
| Control, 3 weeks, 40/75 | 795 | 224 |
| 4% Sodium gluconate, Initial | 1848 | 154 |
| 4% Sodium gluconate, 1 week, 40/75 | 2640 | 250 |
| 4% Sodium gluconate, 3 weeks, 40/75 | 1967 | 128 |
| 4% Sorbitol, Initial | 2265 | 75 |
| 4% Sorbitol, 1 week, 40/75 | 2595 | 200 |
| 4% Sorbitol, 3 weeks, 40/75 | 2659 | 104 |
| 4% Xylitol, Initial | 1343 | 138 |
| 4% Xylitol, 1 week, 40/75 | 2785 | 31 |
| 4% Xylitol, 3 weeks, 40/75 | 2806 | 148 |
| 4% Urea, Initial | 2166 | 206 |
| 4% Urea, 1 week, 40/75 | 2622 | 171 |
| 4% Urea, 3 weeks, 40/75 | 2562 | 333 |
| 4% PG-3, Initial | 2249 | 24 |
| 4% PG-3, 1 week, 40/75 | 2631 | 57 |
| 4% PG-3, 3 weeks, 40/75 | 2841 | 163 |
| 0.25% PVP-K17, Initial | 1371 | 55 |
| 0.25% PVP-K17, 1 week, 40/75 | 1588 | 122 |
| 0.25% PVP-K17, 3 weeks, 40/75 | 1710 | 476 |
| 1.0% PVP-K17, Initial | 2021 | 155 |
| 1.0% PVP-K17, 1 week, 40/75 | 2212 | 165 |
| 1.0% PVP-K17, 3 weeks, 40/75 | 2331 | 108 |
| 4% PVP-10k, Initial | 2488 | 31 |
| 4% PVP-10k, 1 week, 40/75 | 2501 | 110 |
| 4% PVP-10k, 3 weeks, 40/75 | 2034 | 362 |
| 0.25% PVP-K90, Initial | 2705 | 40 |
| 0.25% PVP-K90, 1 week, 40/75 | 1003 | 171 |
| 0.25% PVP-K90, 3 weeks, 40/75 | 1252 | 38 |
| 1.0% PVP-K90, Initial | 1906 | 196 |
| 1.0% PVP-K90, 1 week, 40/75 | 1324 | 103 |
| 1.0% PVP-K90, 3 weeks, 40/75 | 1949 | 77 |

### Example 4

This example utilizes room temperature incubation at ambient humidity to demonstrate improved CHG release of incubated adhesive samples versus control while the protective effect of water-soluble complexing agents continued to remain viable.

Control adhesive was made, coated and laminated as in Example 1. The complexed samples were made by adding the complexing agent to part B, replacing all of the water and part of the isopropanol for the 400 complexing agent samples. Samples with the lower levels of complexing agents had only a portion of the water replaced. A portion of the laminated samples was incubated for 6 weeks at room temperature and all the samples were measured for CHG elution as in Example 1 using an elution time of 30 minutes. The results are provided in Table 6.

TABLE 6
CHG Elution from the samples of Example 4.

| Sample | Eluted CHG (ppm) | S.D. (ppm) |
|---|---|---|
| Control, Initial | 2590 | 52 |
| Control, 6 weeks, RT | 1621 | 471 |
| 4% Sorbitol, Initial | 2265 | 75 |
| 4% Sorbitol, 6 weeks, RT | 2788 | 283 |
| 4% xylitol, Initial | 1343 | 138 |
| 4% xylitol, 6 weeks, RT | 2961 | 123 |
| 0.25% PVP-K17, Initial | 1371 | 55 |
| 0.25% PVP-K17, 6 weeks, RT | 493 | 127 |
| 1.0% PVP-K17, Initial | 2021 | 155 |
| 1.0% PVP-K17, 6 weeks, RT | 2736 | 7 |
| 0.25% PVP-K90, Initial | 2705 | 40 |
| 0.25% PVP-K90, 6 weeks, RT | 2659 | 67 |
| 1.0% PVP-K90, Initial | 1906 | 196 |
| 1.0% PVP-K90, 6 weeks, RT | 2736 | 7 |

### Example 5

This example used the standard acceleration aging conditions utilized for drug products. These accelerated aging conditions predict a shelf-life of 1 year at room temperature when the samples pass testing after 3 months of accelerated aging. The control and complexation agent containing adhesives were made, coated, and laminated as in the previous examples. Accelerated aging was conducted at 40° C./75% R.H. for 3 months. Samples were also removed prior to achieving the accelerated aging conditions by removing them after one and three weeks incubation. Initial (as made) samples, samples exposed to accelerated aging conditions, and samples incubated for one and three weeks were tested for CHG elution as in previous examples using an elution time of 30 minutes. The results in Table 7 clearly show a decrease in elutable CHG at the surface by as much as 95% for the control. The use of xylitol as a complexing agent at both 1% and 2% of the composition exerts an excellent protective effect. Polyglycerol-3 is also shown to act as a protective agent under these conditions.

TABLE 7
CHG elution from samples of Example 5.

| Sample | Eluted CHG (ppm) | S.D. (ppm) |
|---|---|---|
| Control, Initial | 2207 | 72 |
| Control, 1 wk, 40/75 | 1360 | 414 |
| Control, 3 wk, 40/75 | 604 | 166 |
| Control, 3 mo. 40/75 | 137 | 14 |
| 2% PG-3, Initial | 2153 | 1013 |
| 2% PG-3, 1 wk, 40/75 | 1502 | 304 |
| 2% PG-3, 3 wk, 40/75 | 1257 | 227 |
| 2% PG-3, 3 mo., 40/75 | 786 | 201 |
| 1% Xylitol, Initial | 2149 | 841 |
| 1% Xylitol, 1 wk, 40/75 | 2276 | 228 |
| 1% Xylitol, 3 wk, 40/75 | 2282 | 98 |
| 1% Xylitol, 3 mo. 40/75 | 2741 | 257 |
| 2% Xylitol, Initial | 1762 | 852 |
| 2% Xylitol, 1 wk, 40/75 | 2312 | 87 |
| 2% Xylitol, 3 wk, 40/75 | 2318 | 161 |
| 2% Xylitol, 3 mo, 40/75 | 2682 | 97 |

### Example 6

This example probes the effect of multiple ethylene oxide sterilization cycles on the CHG availability at the adhesive surface. Complexed samples were made, coated, and laminated as in earlier examples and subject to 3 commercial ethylene oxide sterilization cycles. The samples were tested for eluted CHG as in previous examples using an elution time of 30 minutes. Results are provided in Table 8.

Note that EO sterilization appears to drastically compromise the protective effect of PVP, but the xylitol and polyglycerol-3 samples remain unaffected.

TABLE 8
CHG elution from the samples of Example 6.

| Sample | | Eluted CHG (ppm) | S.D. (ppm) |
|---|---|---|---|
| Nonsterile | 4% PG-3 | 2217 | 49 |
| samples | 4% Xylitol | 2320 | 373 |
| | 4% PVP K-17 | 1982 | 336 |
| | Control | 2049 | 54 |
| Sterilized | 4% PG-3 | 2160 | 140 |
| samples | 4% Xylitol | 2524 | 372 |
| | 4% PVP K-17 | 1042 | 289 |
| | Control | 1133 | 318 |

1. An antimicrobial-impregnated adhesive sheet, comprising:
an antimicrobial;
a pressure-sensitive adhesive;
a first major surface;
a second major surface; and
a plurality of surface depressions extending inwardly from the first major surface,
wherein an initial available fraction of the antimicrobial at the first major surface is at least 30% of a total amount of the antimicrobial in the antimicrobial-impregnated adhesive sheet, and
wherein the antimicrobial-impregnated adhesive sheet exhibits a retention of the initial available fraction of the antimicrobial at the first major surface, after accelerated aging, of at least 50%.

2. The antimicrobial-impregnated adhesive sheet of claim 1, wherein the plurality of surface depressions are distributed over the first major surface such that a number of surface depressions; per square millimeter; differs by no more than 10%.

3. The antimicrobial-impregnated adhesive sheet of claim 1, wherein the antimicrobial-impregnated adhesive sheet exhibits a rate of antimicrobial release of no more than 10 minutes.

4. The antimicrobial-impregnated adhesive sheet of claim 1, wherein the plurality of surface depressions has an average longest dimension of between 4 and 100 micrometers.

5. The antimicrobial-impregnated adhesive sheet of claim 1, wherein each of the plurality of surface depressions has a spherical shape.

6. The antimicrobial-impregnated adhesive sheet of claim 1, wherein the antimicrobial-impregnated adhesive sheet has a thickness of between 5 and 100 microns.

7. The antimicrobial-impregnated adhesive sheet of claim 1, wherein the pressure sensitive adhesive has an HLB value of less than 5.

8. The antimicrobial-impregnated adhesive sheet of claim 1, wherein the pressure-sensitive adhesive comprises a material selected from the group consisting of an acrylic polymer, an acrylic copolymer, a natural rubber, a synthetic rubber polymer, a synthetic copolymer, and a silicone polymer.

9. A medical article, comprising:
a substrate having a first surface and second surface opposite the first surface; and
the antimicrobial-impregnated adhesive sheet of claim 1 disposed on the first surface.

10. The medical article of claim 9, wherein the substrate is a polymeric film.

11. The medical article of claim 9, further comprising a release liner in contact with the antimicrobial-impregnated adhesive sheet.

12. The medical article of claim 9, wherein the medical article comprises a surgical drape.

13. (canceled)

14. (canceled)

15. (canceled)

16. A method for making an antimicrobial-impregnated adhesive sheet of claim 1, the method comprising:
providing a solvent-based pressure sensitive adhesive solution, comprising a solvent and a pressure sensitive adhesive, wherein the pressure sensitive adhesive has an HLB value of less than 5;
providing an aqueous antimicrobial composition, comprising water, an antimicrobial, and a water-soluble complexing agent, wherein the water is present in the aqueous antimicrobial composition in an amount of 20 weight percent to 80 weight percent of a total weight of the aqueous antimicrobial composition; and
contacting the solvent-based pressure sensitive adhesive solution and the aqueous antimicrobial composition to form an antimicrobial-impregnated adhesive precursor.

17. The method of claim 16, further comprising depositing the antimicrobial-impregnated adhesive precursor on a substrate.

18. The method of claim 17, wherein depositing the antimicrobial-impregnated adhesive precursor occurs within 30 minutes of contacting the aqueous antimicrobial composition and the solvent-based pressure sensitive adhesive solution.

19. The method of claim 16, further comprising drying the antimicrobial-impregnated adhesive precursor to form the antimicrobial-impregnated adhesive sheet.

20. The method of claim 16, wherein the antimicrobial is selected from the group consisting of chlorhexidine gluconate, chlorhexidine acetate, octenidine hydrochloride, polyhexamethylene biguanide salts, a quat ammonium salt, a chlorhexidine salt, a silver salt, a water-soluble iodophor, and triclosan.

21. The method of claim 16, wherein the water-soluble complexing agent is selected from the group consisting of a polyhydroxylated compound, a polymeric polyhydroxylated compound, a carbamide, a lactam, an amide, and combinations thereof.

22. The method of claim 16, wherein the water-soluble complexing agent is present in the aqueous antimicrobial composition in an amount of between 0.1 and 10 wt-%, based on the total weight of the aqueous antimicrobial composition.
